(19) United States
(12) Patent Application Publication
(10) Pub. No.: US 2006/0270053 A1
(43) Pub. Date: Nov. 30, 2006

(54) Apparatus, methods, and systems having gas sensor with catalytic gate and variable bias

(75) Inventors: Vinayak Tilak, Schenectady, NY (US); Kevin Sean Matocha, Rexford, NY (US); Peter Micah Sandvik, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/137,845

(22) Filed: May 26, 2005

(51) Int. Cl. G01N 33/00; G01N 25/26

(52) U.S. Cl. 436/143; 436/160; 977/900

(57) ABSTRACT

According to some embodiments, an electronics based physical gas sensor includes a semiconductor layer, and at least one contact is electrically coupled to the semiconductor layer. A catalytic gate, having a property that changes when the gate is exposed to an analyte, and a variable bias from a voltage source are also provided.

### BACKGROUND

A gas sensor may be used to detect the presence of one or more analytes in a gas. For example, a gas sensor might be used to detect the presence and/or concentration of nitrogen oxides (NO_{X}), which are a group of highly reactive gases that contain varying amounts of nitrogen and oxygen. Such a sensor could be used, for example, to ensure that an industrial process or turbine engine complies with a governmental regulation (e.g., a regulation established by the US Environmental Protection Agency).

A gas sensor may need to selectively detect different species of an analyte. For example, a sensor might need to accurately distinguish between exposure to C₂H₂ and C₂H₄. Moreover, a sensor may need to operate in harsh environments, such as environments having relatively extreme vibration, temperature (e.g., 600° C.), chemical and/or pressure conditions. Also note that it may be impractical to use a sensor if it is too large, expensive, or unreliable.

### SUMMARY

According to some embodiments, an electronics based physical gas sensor includes a semiconductor layer, and at least one contact is electrically coupled to the semiconductor layer. A catalytic gate, having a property that changes when the gate is exposed to an analyte, and a variable bias from a voltage source are also provided.

### BRIEF DESCRIPTION OF THE DRAWINGS

FIG. 1 is a block diagram overview of an electronics based physical gas sensor.

FIG. 2 is side view of a FET based physical gas sensor with a catalytic gate.

FIG. 3 illustrates a method of detecting an analyte according to an exemplary embodiment of the invention.

FIG. 4 is a graph illustrating current between a source and a drain over time according to an exemplary embodiment of the invention.

FIG. 5 is a gas sensor with a catalytic gate and an alternating current bias according to an exemplary embodiment of the invention.

FIG. 6 is a gas sensor wherein a catalytic material acts as a resistor according to another exemplary embodiment of the invention

FIG. 7 is a side view of a capacitor-based gas sensor according to an exemplary embodiment of the invention.

FIG. 8 is a perspective view of a capacitor-based gas sensor according to an exemplary embodiment of the invention.

FIG. 9 is a side view of a gas sensor with multiple catalytic gates according to an exemplary embodiment of the invention.

FIG. 10 is a gas sensor with multiple catalytic gates and a shielding layer according to an exemplary embodiment of the invention.

FIG. 11 is a schematic view of a gas sensor with multiple catalytic gates and associated voltage dividing resistors according to an exemplary embodiment of the invention.

FIG. 12 is a schematic view of a gas sensor with multiple drains and associated voltage dividing resistors according to an exemplary embodiment of the invention.

FIG. 13 is a system in accordance with an exemplary embodiment of the invention.

### DETAILED DESCRIPTION OF EXEMPLARY EMBODIMENTS

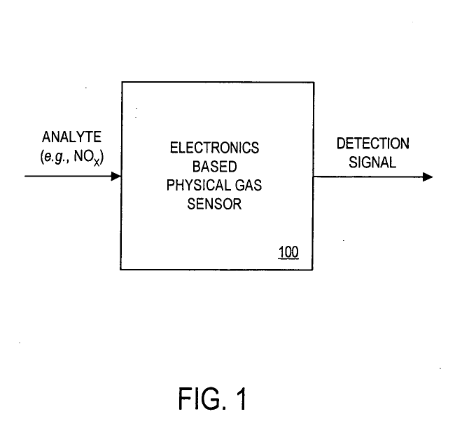

A gas sensor may be used to determine if an “analyte” is present and/or to quantify an amount of the analyte. As used herein, the term “analyte” may refer to any substance to be detected and/or quantified, including a gas, a vapor, and/or a bioanalyte. For example, FIG. 1 is a block diagram overview of an electronics based physical gas sensor 100 that may be used to detect whether or not an analyte is present (and/or to determine a concentration of the analyte). The sensor 100 might be, for example, an in-situ sensor that directly samples an airstream to be analyzed. In this way, the sensor 100 can be exposed to the airstream and generate a detection signal indicating whether or not a particular analyte is present (e.g. whether or not the amount of nitrogen oxide in the surrounding atmosphere exceeds a pre-determined level). The sensor 100 can also generate a signal proportional to the concentration of the analyte and thereby measure the concentration of the analyte.

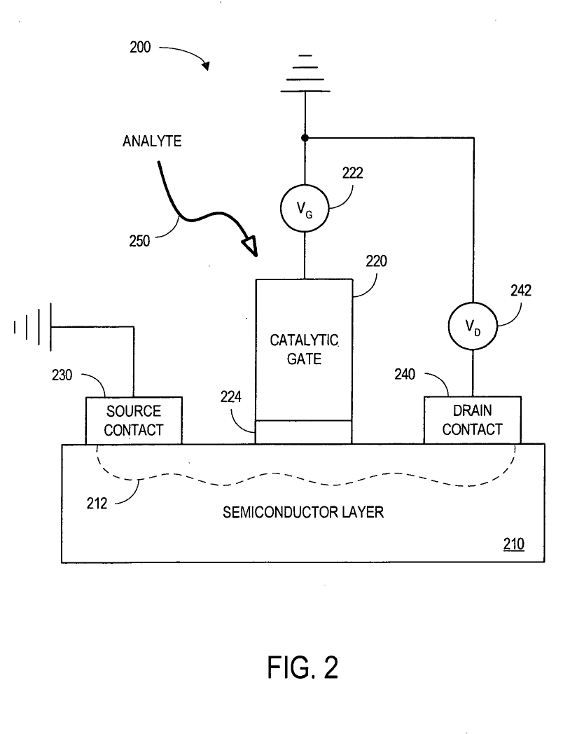

A sensor may use a catalytic material to facilitate detection of an analyte. For example, FIG. 2 is side view of a gas sensor 200 that includes a semiconductor layer 210. The semiconductor layer 210 might be, for example, a Silicon Carbide (SiC) and/or GaN substrate.

A dielectric layer 224 separates a catalytic gate 220 from the semiconductor layer 210. The dielectric layer 224 may comprise, for example, a layer of SiO₂, SiN, HfO₂ and/or a metal oxide or any combination thereof. The catalytic gate 220 may be, for example, a metallic contact. In this way, the gate 220 and dielectric layer 224 might form, for example, a metal/metal oxide stack on silicon nitride. A gate voltage source 222 may provide a fixed gate voltage V_{G} to the catalytic gate 220 (e.g., on the side of the gate 220 opposite the dielectric layer 224 and the semiconductor layer 210).

A source contact 230 is electrically coupled to the semiconductor layer 210 and to ground. Similarly, a drain contact 240 is coupled to the semiconductor layer 210, remote from the source contact 230, as well as a drain voltage source 242. The drain voltage source 242 provides a fixed drain voltage V_{D} to the drain contact 240. The source contact 230 and the drain contact 240 may comprise, for example, ohmic contacts made of nickel or aluminum.

The arrangement illustrated in FIG. 2 may comprise a three-terminal Field Effect Transistor (FET). For example, a source drain current may flow through a channel region 212 between the source contact 230 and the drain contact 240. Moreover, the catalytic gate 220 may be used to influence the region 212 and change the source drain current (e.g., by restricting the region 212 and reducing the current or expanding the region 212 and increasing the current). Note that a relatively small change associated with the gate might result in a relatively large change in the source drain current.

According to some embodiments, the sensor 200 is fabricated on a Wide Bandgap (WBG) semiconductor. For example, the catalytic gate 220, dielectric layer 224, and semiconductor layer 210 might be associated with a heterojunction wherein a surface of a heavily doped/high bandgap material interfaces with a surface of a lightly doped/low bandgap material. This heterojunction may be associated with a Schottky contact.

The gate 220 may be “catalytic” in that a property of the gate 220 changes when exposed to an analyte 250 (like hydrogen or NO_{X}). For example, molecules of the analyte 250 may diffuse through the gate 220 and adsorb at the metal-dielectric interface. The adsorbed molecules may cause a change in the effective Schottky barrier height (and the direction and/or quantity of the change might depend on the amount and/or type of analyte that is adsorbed). This change in the Schottky barrier height may, for example, change the capacitance of the gate 220 and/or influence the region 212. The resulting change in current through the region 212 may then be correlated to the concentration of the analyte 250 in the sensor's environment.

Although the sensor 200 may be used to detect the presence of the analyte 250, it might be difficult to use the sensor 200 to selectively detect different types or species of analyte.

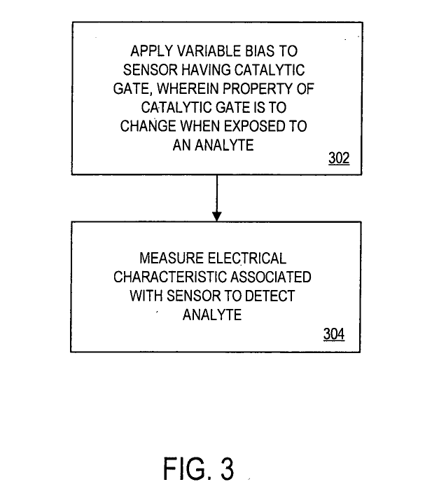

FIG. 3 illustrates a method of detecting an analyte according to an exemplary embodiment of the invention. At Step 302, a variable bias is applied to a sensor having a catalytic gate (that is, a property of the catalytic gate will change when the gate is exposed to an analyte).

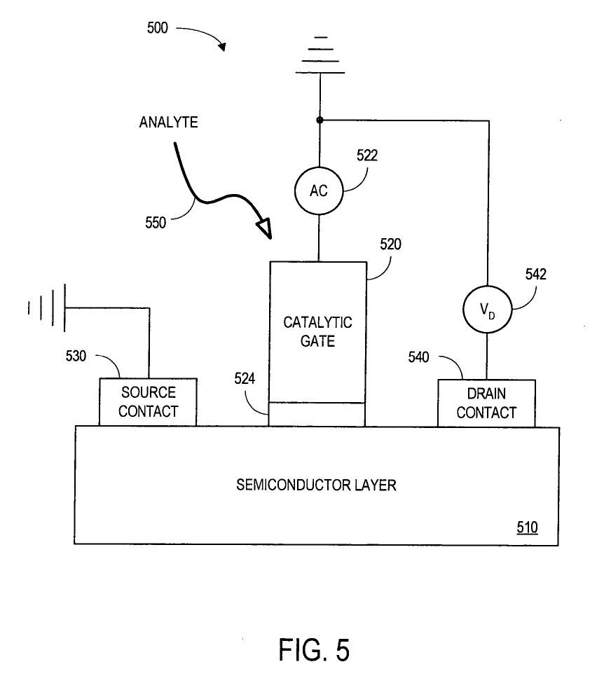

As used herein, a bias may be “variable” in that, for example, the bias changes over time—such as when an Alternating Current (AC) bias is provided to a sensor's gate contact. A bias might also be “variable” in that a first bias is applied to detect a first species of analyte while a second bias is later applied to the same sensor (e.g., to detect a second species of analyte). As another example, a first bias might be applied to a first sensor (or sub-sensor) while a second bias is applied to a second sensor at the same time (e.g., so that multiple species of analyte can be detected simultaneously). Several examples of variable biases are provided in connection with FIGS. 5-12.

At Step 304, an electrical characteristic associated with the sensor is measured to detect the analyte. The electrical characteristic might be associated with, for example, a source drain current level. As another example, the characteristic might be associated with a source drain current waveform. For example, a frequency and/or a time constant of a response signal waveform might be monitored to determine information associated with background concentrations of an analyte or other substance.

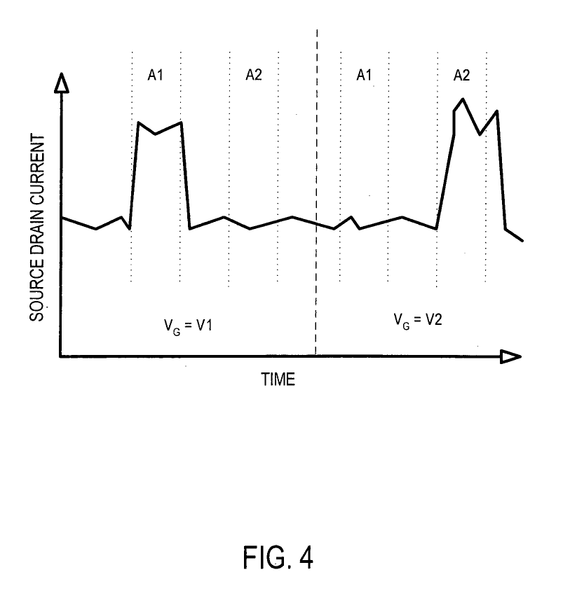

FIG. 4 is a graph illustrating source drain current over time according to an exemplary embodiment of the invention. Initially, a first gate voltage (V1) is applied to a sensor. In this case, the presence of a first species of analyte (A1) increases the source drain current while the presence of a second species of analyte (A2) does not. A second gate voltage (V2) is then applied to the same sensor. Now exposing the sensor to A1 does not alter the current, but exposing the sensor to A2 does. Thus, a single sensor may be used to selectively detect either species of analyte depending on the bias that is applied sensor's gate.

FIG. 5 is a gas sensor 500 that may be used to detect an analyte 550 according to an exemplary embodiment of the invention. Although NO_{X} is used as an example with respect to some of the embodiments described herein, note that a sensor may be used to detect other analytes, such as, for example, CO_{X}, SO_{X}, NH₃, O₂, CH₄, C₂H₂, C₂H₄, and/or H₂.

The sensor 500 includes a semiconductor layer 510, such as layer that includes silicon carbide, gallium nitride, and/or a WBG material. According to some embodiments, the layer 530 includes a metal, such as aluminum, gold, nickel, rhenium, tantalum, and or osmium. Moreover, according to some embodiments, the layer 520 is formed from a metal oxide such as gallium oxide, silver oxide, indium oxide, vanadium oxide, Mn₂O₃, CuO, Cr₂O₃, Co₂O₃, ZnO, Ge₂O₃, FeO₂, and/or bismuth molybdate. According to other embodiments, the layer 520 is formed from a metal alloy, such as platinum/rhodium, palladium/iridium, platinum/titanium/gold, platinum/ruthenium, platinum/iridium, and/or platinum/gold.

A dielectric layer 524 separates a catalytic gate 520 from the semiconductor layer 510. The dielectric layer 524 might be, for example, a layer of SiO₂, SiN, HfO₂ and/or a metal oxide. The catalytic gate 520 may be, for example, a metallic contact such as one formed from a combination of oxides including platinum/tin oxide, platinum/indium oxide, zinc oxide/vanadium oxide, indium oxide/tin, or oxide/manganese oxide, Pt/Ga₂O₃, Pt/Ag/Ga₂O₃. According to some embodiments, the catalytic gate 520 comprises a material of the formula ABO₃ where A is lanthanum and B is any transition metal or alkaline earth metal. In this way, the gate 520 and dielectric layer 524 might form, for example, a metal/metal oxide stack on silicon nitride.

Note that the catalytic gate 520 may be a multiple layer stack of catalytic material layers. Each layer might include, for example, a single catalytic material or a combination alloy of catalytic materials. According to some embodiments, each layer of material may have a thickness from about 50 Å to about 8000 Å.

A source contact 530 is electrically coupled to the semiconductor layer 510 and an electrical ground. Similarly, a drain contact 540 is coupled to the semiconductor layer 510, remote from the source contact 530, as well as a drain voltage source 542. The drain voltage source 542 provides a drain voltage V_{D} to the drain contact 540. The source contact 530 and the drain contact 540 may be formed using, for example, nickel, titanium, aluminum, gold, chromium, and/or indium.

The arrangement illustrated in FIG. 5 may comprise a three-terminal FET device. For example, a source drain current may flow through a channel region between the source contact 530 and the drain contact 540. Moreover, the catalytic gate 520 may be used to influence the region 512 and change the source drain current (e.g., by restricting the region and reducing the current or expanding the region and increasing the current). Note that a relatively small change in the concentration of adsorbed analyte molecules in catalytic gate might result in a relatively large change in the source drain current.

According to some embodiments, the sensor 500 acts as a WBG based FET device. For example, the catalytic gate 520, dielectric layer 524, and semiconductor layer 510 might be associated with a heterojunction wherein a surface of a heavily doped/high bandgap material interfaces with a surface of a lightly doped/low bandgap material.

A property of the catalytic gate 520 may change when exposed to an analyte 550. For example, when the gate 520 is exposed to an analyte 550, molecules of the analyte may diffuse through the gate 520 and adsorb at the metal-semiconductor interface. The adsorption of the analyte by the catalytic gate 520 might, for example, change its capacitance and create a layer of ions between the catalytic gate 520 and a dielectric interface. This change may also change the capacitance of the gate 520 and/or influence a channel formed between the source contact 530 and the drain contact 540. The resulting change in current through the channel may then be correlated to the concentration of the analyte 550.

According to this embodiment, an AC voltage source 522 provides a bias that varies over time to the catalytic gate 520 (e.g., on the side of the gate 520 opposite the dielectric layer 524 and the semiconductor layer 510). Note that the AC bias may cause the adsorbed molecules in the gate 520 to move closer or further from the catalyst gate into the dielectric layer. Moreover, different types of molecules may move further up and down as compared to other molecules (e.g., based on the weight, mobility, and/or charge of each type of molecule) and the average displacement of a particular type of molecule might be based on the Root Mean Squared (RMS) value of the AC signal. In this way, applying an AC frequency to the sensor may improve the ability of the sensor 500 to detect a particular species of analyte (e.g., because other species may be moved further away from the junction).

According to some embodiments, the frequency associated with the AC voltage source 522 is varied to adjust the selectivity of the sensor 500 to different species of analyte. For example, a first AC frequency might be applied (and the source drain current monitored) to detect a first species of analyte while a second AC frequency might be used to detect a second species of analyte.

Although an AC bias is described with respect to FIG. 5, note that varying levels of a DC bias might be used to achieve a similar result. For example, a first DC bias level might influence one type of adsorbed molecule more than other types (and a second DC bias level might have a similar impact on a different type). The resulting changes in the catalytic gate 520 and the source drain current may then be used to detect different species of analyte 550.

Moreover, although a variable bias is applied to the catalytic gate 520 in FIG. 5, note that a variable bias may be applied to any terminal of the FET device. For example, a bias that varies over time might be applied to the drain contact 542 while a constant bias is applied to the gate 520. As another approach, the biases that are applied to both the gate 520 and the drain contact 540 might be varied.

As still another example, the FET device might be operated in a constant source drain current mode while the threshold value of the device is monitored (e.g., the level at which the device will turn “on” or “off”). A change in the threshold value may then be correlated to a concentration of analyte. Note that this mode of operation might be associated with constant power dissipation (and hence constant temperature operation).

According to some embodiments, an additional passivation layer is applied to a portion of the surface of the semiconductor layer 510. The passivation layer may comprise, for example, MgO, Sr₂O₃, ZrO2, Ln₂O₃, TiO₂, AlN, and/or carbon and may act to improve the thermal stability and reproducibility of the sensor 500.

According to some embodiments, a heater may be provided proximate to the catalytic gate 520. The heater might comprise, for example, a wire of titanium and/or nickel and may be used to hold the device to a substantially constant temperature during operation. Such an approach might reduce any drift in operation of the sensor 500 due to changes in temperature. Another approach is to attach the die onto a ceramic board and deposit a metal line of Ti/Au on the backside to heat the device/

According to some embodiments, a “reset” signal may be applied to the sensor 500. Consider, for example, a catalytic gate 520 that has been exposed to (and therefore adsorbed) an analyte. In this case, a bias could be applied to the catalytic gate 520 in order to facilitate the expulsion of any adsorbed molecules (e.g., and reduce the device's “memory” that it was exposed to the analyte). Such a reset pulse might be applied, for example: periodically; after a threshold amount of an analyte has been detected; and/or when a different species of analyte is to be sensed by the sensor 500. Note that the polarity and magnitude of the reset signal may determine which types of analytes are expelled from the catalytic gate 520.

Note that a sensor might be creating using any type of FET device, including a Metal Oxide Semiconductor FET (MOSFET), a Heterostructure FET (HFET), and/or a Metal-Insulator Semiconductor Heterostructure FET (MISHFET).

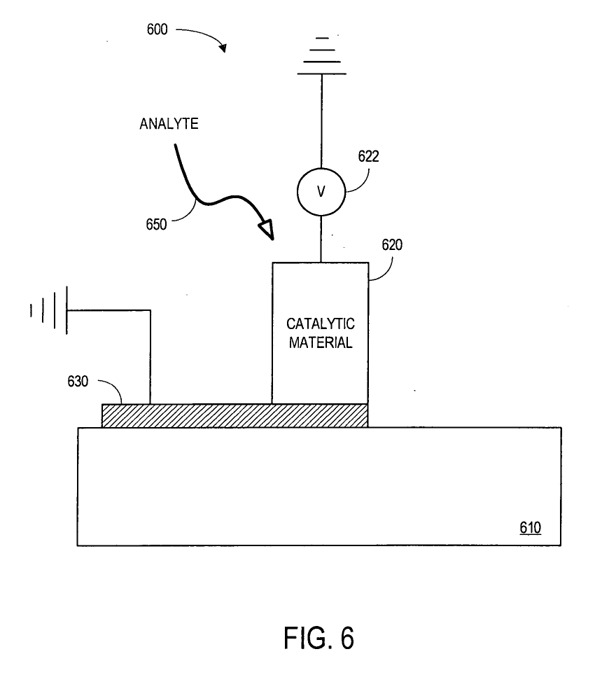

Moreover, a sensor might be implemented using a device other than a transistor. For example, FIG. 6 is a gas sensor 600 wherein a gate of catalytic material 620 acts as a resistor according to another exemplary embodiment of the invention. In particular, a conducting layer 630 on a substrate 610 couples the catalytic material 620 to ground. The catalytic material might comprise, for example, any of the materials discussed with respect to FIG. 5. A voltage source 622 may be used to provide a variable bias to the catalytic material 620. In this case, the impedance of the catalytic material 620 might change when it is exposed to an analyte 650. Moreover, the bias provided by the voltage source 622 may determine how different species of analyte will change the catalytic material's resistance. As a result, the current through the catalytic material 620 and/or the conducting layer 630 may be monitored to detect the presence of a particular species of analyte. Note that such an approach might be used in combination with the approach described with respect to FIG. 5 (and the two different methods may be used to independently measure and verify analyte concentration).

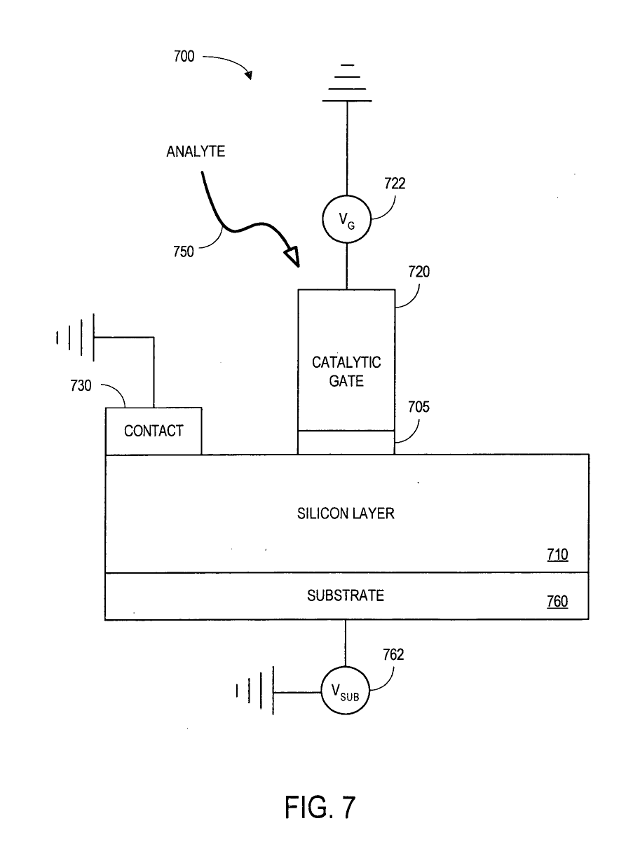

According to another embodiment, a capacitor may be used to detect an analyte. For example, FIG. 7 is a side view of a capacitor-based gas sensor 700 according to an exemplary embodiment of the invention. In this case, a catalytic gate 720 is formed on a top surface of semiconductor layer 710 along with a ground contact 730. Moreover, a dielectric passivation layer 705 may be provided atop the semiconductor layer 710 and beneath the catalytic gate 720. A gate voltage source 722 provides a voltage (V_{G}) to the catalytic gate 720. According to this embodiment, a substrate 760 is formed on a bottom surface of the semiconductor layer 710 and a substrate voltage source 762 applies a substrate bias (V_{SUB}) to the substrate 760. In this way, the capacitance characteristics of the device may be altered when the catalytic gate 720 adsorbs molecules of an analyte 750. As a result, a body current running through the semiconductor layer 710 could be measured to detect the analyte. According to some embodiments, the semiconductor layer 710 is grown on the substrate 760.

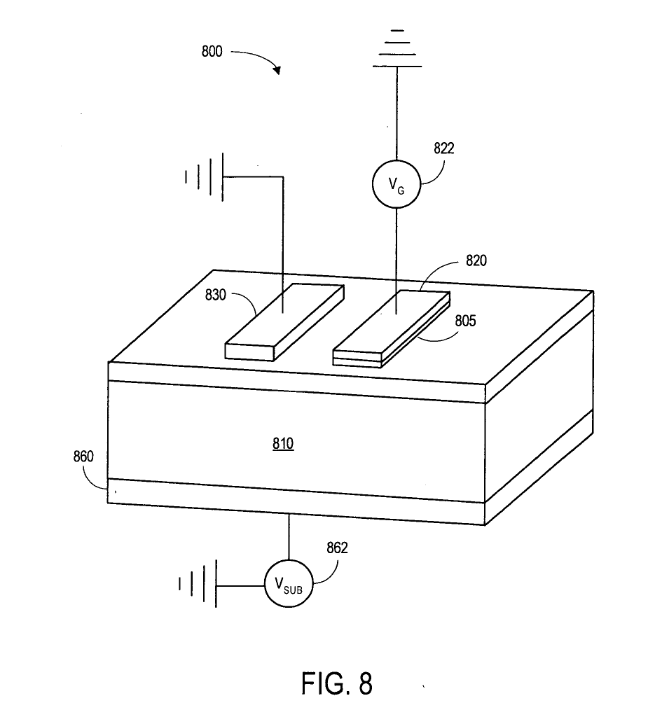

FIG. 8 is a perspective view of a capacitor gas sensor 800 according to an exemplary embodiment of the invention. As before, a catalytic gate 820 is formed on a top surface of semiconductor layer 810 along with a ground contact 830, and a gate voltage source 822 provides a voltage (V_{G}) to the catalytic gate 820. According to some embodiments, a dielectric passivation layer 805 is provided atop the semiconductor layer 810 and beneath the catalytic gate 820. A substrate 860 is formed on a bottom surface of the semiconductor layer 810 and a substrate voltage source 862 applies a substrate bias (V_{SUB}) to the substrate 860. When the catalytic gate 820 adsorbs molecules of an analyte, the capacitance of the device will change, and its steady state capacitance, or small signal capacitance may be measured to detect the analyte.

According to some embodiments, a similar substrate and/or substrate bias may be combined with the approach described with respect to FIG. 5. For example, the source drain current might be monitored to detect an analyte and the body current might be used to determine an existing temperature of the device.

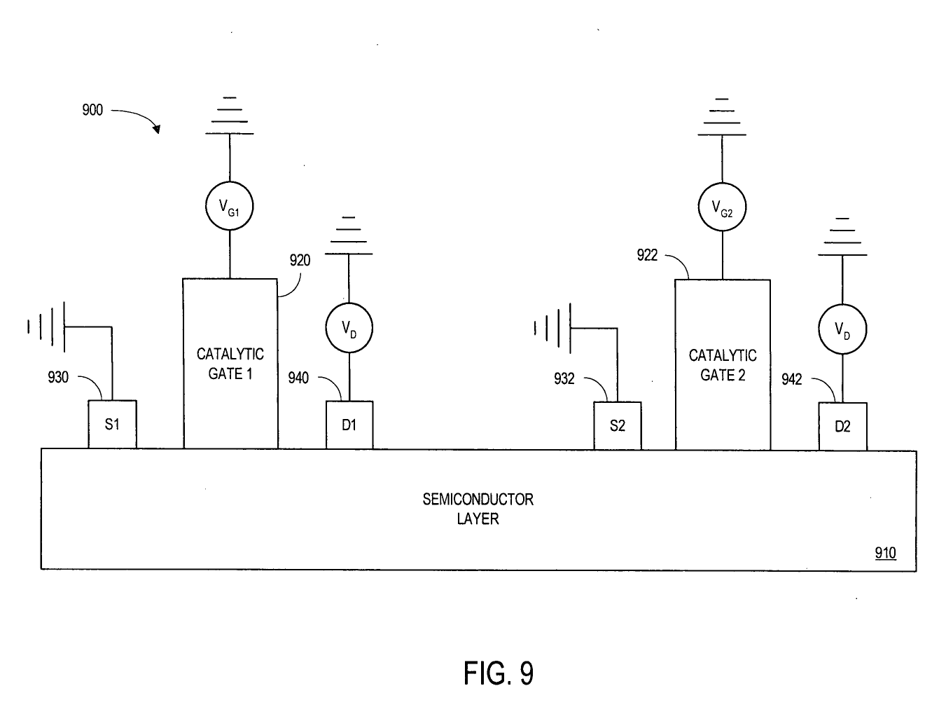

Instead of (or in addition to) providing a bias the varies dynamically over time, according to some embodiments different biases may be simultaneously provided for different sensors or sub-sensors. For example, FIG. 9 is a side view of a gas sensor 900 with multiple catalytic gates 920, 922 according to an exemplary embodiment of the invention. In particular, the sensor 900 includes a first FET device comprising the first catalytic gate 920, a first source contact 930 and a first drain contact 940 formed on a semiconductor layer 910. Similarly, the second catalytic gate 922, a second source contact 932, and a second drain contact 942 formed on the layer 910 comprise a second, independent FET device.

A first gate voltage source provides a first gate voltage (V_{G1}) to the first catalytic gate while a second gate voltage source provides a second t gate voltage (V_{G2}) to the second catalytic gate (and V_{G1} does not equal V_{G2}). By providing different biases to the gates 920, 922, the sensor may be used to detect multiple species of analytes. Although two FET devices are illustrated in FIG. 9, note that embodiments may include any number of the devices disclosed herein. Moreover, a sensor might include different types of devices. For example, a sensor might include both an enhancement mode FET and a depletion mode FET.

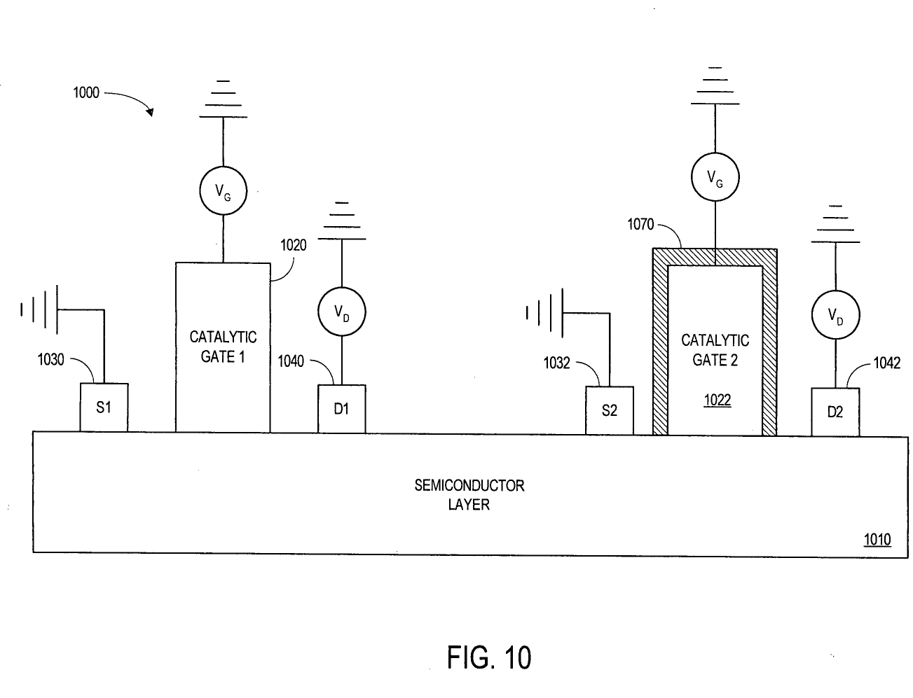

According to some embodiments, one or more devices in an array are prevented from adsorbing the analyte. For example, FIG. 10 is a sensor 1000 that includes a first FET device with first catalytic gate 1020, a first source contact 1930 and a first drain contact 1040 formed on a semiconductor layer 1010. A second catalytic gate 1022, a second source contact 1032, and a second drain contact 1042 are also formed on the layer 1010 to provide a second, independent FET device.

In this case, a shielding layer 1070 is formed on the second catalytic gate 1922 to prevent it from being exposed to an analyte. The shielding layer 1070 might include, for example, silicon dioxide, silicon nitride and/or hafnium dioxide that will block molecules of analyte from being adsorbed by the second catalytic gate 1022. In this way, the source drain current associated with the first FET device might be monitored to detect a change in analyte concentration while the source drain current associated with the second FET device might be monitored to detect a change in, for example, a temperature.

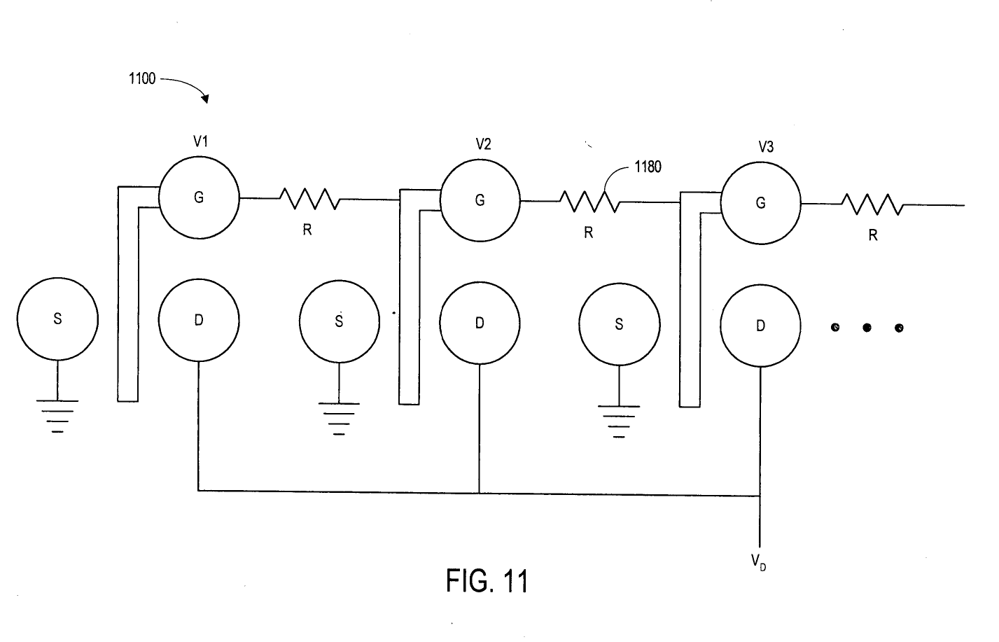

According to some embodiments, a single voltage source may be used to provide variable biases for a sensor. For example, FIG. 11 is a schematic view of a gas sensor 1100 with multiple catalytic gates and associated voltage dividing resistors 1180 (R) according to an exemplary embodiment of the invention. According to this embodiment, three different FET devices are provided (e.g., to detect three different species of analyte or two different species of analyte along with a temperature information). As a result of the three voltage dividing resistors 1180, a single voltage source may be used to provide the three catalytic gates with three different voltage levels (V1, V2, V3). Although the three resistors 1180 illustrated in FIG. 11 have the same resistance R, note that different levels of resistance could be provided as appropriate.

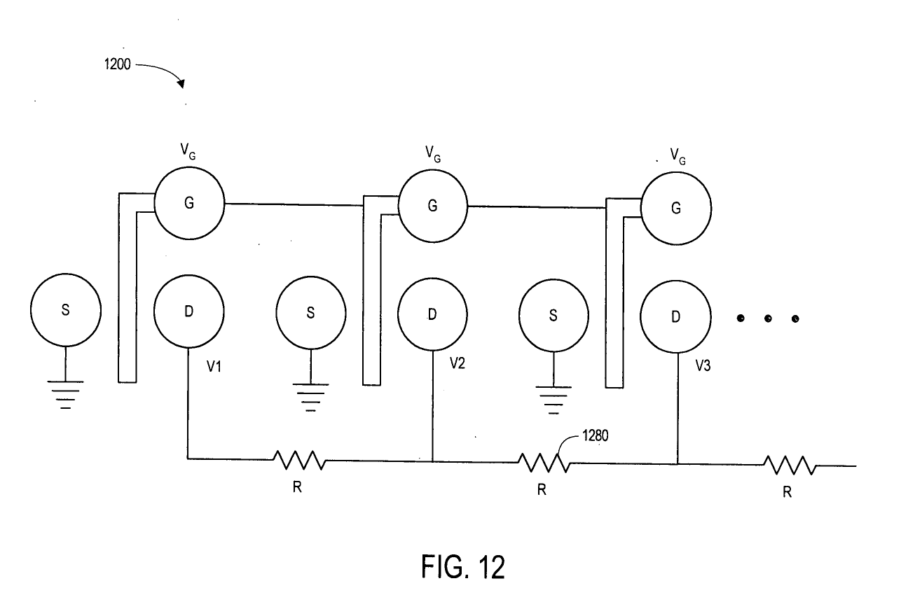

Instead of (or in addition to) providing different biases to different catalytic gates, a sensor array could provide variable biases to source or drain contacts. For example, FIG. 12 is a schematic view of a gas sensor 1200 with multiple drains and associated voltage dividing resisters 1280 (R) according to an exemplary embodiment of the invention. As before, three different FET devices are provided. In this case, however, the three voltage dividing resistors 1280 let a single voltage source provide the three drains with three different voltage levels (V1, V2, V3).

Accordingly, embodiments described herein may provide sensors that are able to selectively detect different species of an analyte. Moreover, the sensors may appropriate for use in systems associated relatively harsh environments.

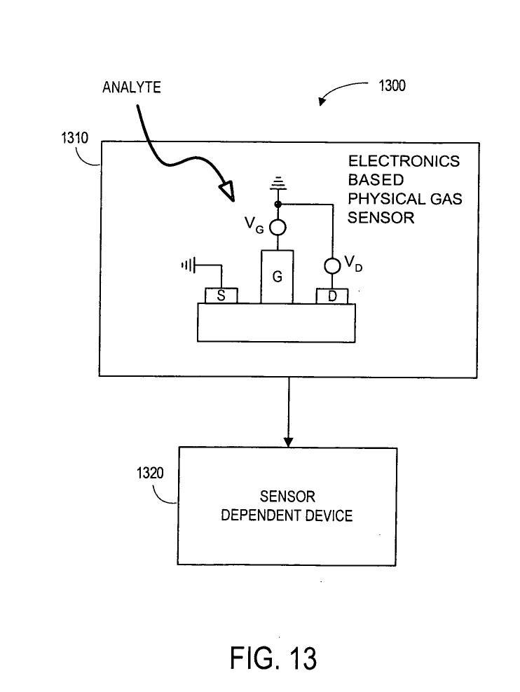

For example, FIG. 13 is a system 1300 in accordance with an exemplary embodiment of the invention. The system 1300 includes a electronics based physical gas sensor 1310 according to any of the embodiments described herein. For example, the sensor might include a wide bandgap semiconductor layer, a contact electrically coupled to the semiconductor layer, an insulating layer formed on the semiconductor layer, a catalytic gate formed on the insulating layer, and a voltage source to provide a bias that is at least one of: (i) variable over time, or (ii) variable between sensors or devices within the sensor 1310. According to some embodiments, the sensor 1310 is a physical gas sensor device.

Note that wide bandgap material may be capable of withstanding the temperatures and corrosive conditions associated with harsh environments. For example, the materials may provide chemically stable, thermally stable, repeatable responses in wide temperature and pressure ranges. Moreover, such materials may be cost effective in that they might be manufactured into devices on a relatively large scale along the lines of well-established semiconductor devices. Note that computer programming or similar techniques may be used to adjust voltage levels and/or monitor characteristics for the sensor 1310 as appropriate.

According to some embodiments, the sensor 1310 is encapsulated. The encapsulation might, for example, protects the sensor 1310 from high temperatures and/or corrosive atmospheres. The encapsulant might, for example, cover the ohmic contact metals and peripheral areas of the sensor 1310 which do not benefit from exposure to the gases. This coverage may also be enhanced by forming a bond with the underlying layer which does not permit the flow of gases or other corrosive molecules which would be a detriment to the sensor 1310 over time. Examples of suitable materials for encapsulating include, but are not limited to, silicon carbide, ceramic-based epoxies such as those containing alumina, glass, quartz, silicon nitride, and/or silicon dioxide. The encapsulation layer might be deposited by any method, such as Plasma Enhanced Chemical Vapor Deposition (PECVD) or Low Pressure Chemical Vapor Deposition (LPCVD). Of course, at least a portion of one or more catalytic gate electrodes will remain exposed to ambient gases.

The system also includes a sensor dependent device 1320. The sensor dependent device 1320 might be associated with, for example, an air quality device, an oil quality device, an industrial process control device, an emissions management device, and/or a turbine sensor.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed:

1. An electronics based physical gas sensor, comprising:
a semiconductor layer;
at least one contact electrically coupled to the semiconductor layer;
a catalytic gate, wherein a property of the catalytic gate is to change when the gate is exposed to an analyte; and
a voltage source to provide a variable bias.

2. The sensor of claim 1, wherein the variable bias is associated with a selectivity of the sensor to the analyte.

3. The sensor of claim 1, wherein adsorption of the analyte by the catalytic gate changes its Schottky barrier height and creates a layer of ions between the catalytic gate and a dielectric interface.

4. The sensor of claim 1, wherein adsorption of the analyte by the catalytic gate changes its capacitance.

5. The sensor of claim 1, wherein the voltage source is to provide the variable bias to at least one of: (i) the catalytic gate, or (ii) a drain contact electrically coupled to the semiconductor layer.

6. The sensor of claim 1, wherein the voltage source is to provide a bias that varies dynamically over time.

7. The sensor of claim 6, wherein the contact is a source contact electrically coupled to ground, and further comprising:
a dielectric layer between a surface of the semiconductor layer and the catalytic gate; and
a drain contact electrically coupled to the semiconductor layer and a drain voltage source.

8. The sensor of claim 6, wherein the catalytic gate is to influence a channel between the drain contact and the source contact when exposed to the analyte.

9. The sensor of claim 6, wherein the voltage source is to provide a first bias associated with a first analyte and a second bias associated with a second analyte.

10. The sensor of claim 12, wherein the source contact, drain contact, and catalytic gate are associated with: (i) a metal oxide semiconductor field effect transistor, (ii) a heterostructure field effect transistor, or (iii) a metal-insulator semiconductor heterostructure field effect transistor.

11. The sensor in claim 1 wherein the ohmic contact, a catalytic gate contact over a dielectric layer on top of a semiconductor, fabricated to form a capacitor.

12. The sensor of claim 6, wherein the dielectric layer comprises at least one of: (i) silicon dioxide, (ii) silicon nitride, or (iii) hafnium oxide.

13. The sensor of claim 6, wherein the voltage source is to provide an alternating bias to the catalytic gate.

14. The sensor of claim 6, wherein the alternating bias is to have a variable frequency.

15. The sensor of claim 1, wherein the catalytic gate is a first catalytic gate, and further comprising:
a second catalytic gate, wherein the voltage source is to provide a first bias associated with the first catalytic gate that varies from a second bias associated with the second catalytic gate.

16. The sensor of claim 15, wherein the first bias is to be provided to the first catalytic gate and the second bias is to be provided to the second catalytic gate.

17. The sensor of claim 15, wherein the first bias is to be provided to a first drain associated with the first catalytic gate and the second bias is to be provided to a second drain associated with the second catalytic gate.

18. The sensor of claim 15, further comprising:
a voltage divider to provide the first bias and the second bias.

19. The sensor of claim 15, wherein the first catalytic gate is to sense a first analyte and the second catalytic gate is to sense a second analyte.

20. The sensor of claim 15, further comprising:
a passivating layer comprising of silicon nitride or hafnium oxide or silicon dioxide or any combination thereof to prevent the second catalytic gate from being exposed to the analyte.

21. The sensor of claim 15, wherein the first catalytic gate is associated with an enhancement mode field effect transistor and the second catalytic gate is associated with a depletion mode field effect transistor

22. The sensor of claim 1, wherein the contact and the catalytic gate are proximate to a top surface of the semiconductor layer, and further comprising:
a substrate on which the semiconductor is grown and forms the bottom surface.

23. The sensor of claim 1, wherein a substrate bias is applied to the substrate.

24. The sensor of claim 1, wherein the analyte comprises at least one of: NO_{X}, COₓ, SOₓ, NH₃, O₂, CH₄, C₂H₂, C₂H₄ or H₂.

25. The sensor of claim 1, wherein the semiconductor layer comprises at least one of: (i) silicon carbide, (ii) group III nitride like Gallium Nitride, Aluminum Nitride or Indium Nitride or any alloy of these semiconductors, (iii) any semiconductor with a bandgap of greater than 2 eV, (iv) a metal oxide.

26. The sensor of claim 1, wherein the catalyst gate material includes a: platinum, ruthenium, silver, palladium, iridium, indium, rhodium, titanium, gold, rhenium, tantalum, osmium, gallium oxide, silver oxide, indium oxide, vanadium oxide, Mn₂O₃, CuO, Cr₂O₃, Co₂O₃, ZnO, Ge₂O₃, FeO₂, or bismuth molybdate or any combination thereof It may also include a material of formula ABO₃ where A is lanthanum and B is any transition metal or alkaline earth metal.

27. The sensor of claim 1, further comprising a heater.

28. The sensor of claim 1, wherein the sensor is a physical gas sensor system device.

29. A method, comprising:
applying a variable bias to a sensor having a catalytic gate, wherein a property of the catalytic gate changes when the gate is exposed to an analyte; and
measuring an electrical characteristic associated with the sensor to detect the analyte.

30. The method of claim 29, wherein the variable bias is applied to at least one of: (i) the catalytic gate, or (ii) a drain contact of the sensor.

31. The method of claim 29, wherein the electrical characteristic is associated with at least one of: (i) a source drain current, (ii) a gate current, (iii) a body current, (iv) a threshold voltage, (v) a frequency of a response signal waveform, or (vi) a time constant of a response signal waveform.

32. The method of claim 29, wherein said applying comprises:
applying a specific bias to improve detection of a particular analyte.

33. The method of claim 29, wherein said applying comprises:
applying an alternating current having a first frequency to detect a first species of analyte; and
applying an alternating current having a second frequency to detect a second species of analyte.

34. The method of claim 29, further comprising:
applying a reset signal to expel the analyte from the catalytic gate.

35. A system, comprising:
a gas sensor, including:
a wide bandgap semiconductor layer,
a contact electrically coupled to the semiconductor layer,
an insulating layer formed on the semiconductor layer,
a catalytic gate formed on the insulating layer, and
a voltage source to provide a bias that is at least one of: (i) variable over time, or (ii) variable between sensors or (iii) having a variable frequency;
a sensor dependent device.

36. The system of claim 35, wherein the sensor dependent device is associated with at least one of: (i) an air quality device, (ii) an oil quality device, (iii) an industrial process control device, (iv) an emissions management device, or (v) a turbine sensor.
